(19) United States
(12) Patent Application Publication
(10) Pub. No.: US 2022/0356291 A1
(43) Pub. Date: Nov. 10, 2022

(54) ACID-BLOCKED PYRROLIDINE CATALYSTS FOR POLYURETHANE FOAM

(71) Applicant: Huntsman Petrochemical LLC, The Woodlands, TX (US)

(72) Inventors: DiAnne Pham, The Woodlands, TX (US); Matthew T. Meredith, The Woodlands, TX (US); Robert A. Grigsby, The Woodlands, TX (US)

(73) Assignee: Huntsman Petrochemical LLC, The Woodlands, TX (US)

(21) Appl. No.: 17/627,387

(22) PCT Filed: Jul. 14, 2020
(86) PCT No.: PCT/US2020/041897

§ 371 Date: Jan. 14, 2022

Related U.S. Application Data
(60) Provisional application No. 62/875,629, filed on Jul. 18, 2019.

(51) Int. Cl. C08G 18/20; C08G 18/08; C08G 18/48; C08J 9/14

(52) U.S. Cl. CPC C08G 18/2081; C08G 18/14; C08G 18/4812; C08G 18/4829; C08J 9/144; C08G 2101/00

(57) ABSTRACT

The present disclosure relates to acid-blocked pyrrolidine catalysts for use in a polyurethane formulation. The polyurethane formulation includes the acid-blocked pyrrolidine catalyst, a compound containing an isocyanate functional group, an active hydrogen-containing compound and a halogenated olefin compound. The use of such acid-blocked pyrrolidine catalysts show surprisingly low reactivity with halogenated olefin compounds yet sufficient reactivity to catalyze polyurethane formation.

### CROSS-REFERENCE TO RELATED APPLICATION

This application is the National Phase of International Application PCT/US2020/041897 filed Jul. 14, 2020 which designed the U.S. and claims priority to U.S. Provisional Patent Application 62/875,629 filed Jul. 18, 2019. The noted applications are incorporated herein by reference.

### STATEMENT REGARDING FEDERALLY SPONSORED RESEARCH OR DEVELOPMENT

Not applicable.

### FIELD

The present disclosure generally relates to acid-blocked pyrrolidine catalysts for use in the production of flexible and rigid polyurethane foam and other polyurethane materials.

### BACKGROUND

Polyurethane foams are widely known and used in a variety of applications, such as in the automotive and housing industry. These foams are produced by the reaction of a polyisocyanate with a polyol in the presence of various additives. One such additive is an amine catalyst which is used to accelerate blowing (the reaction of water with polyisocyanate to generate CO₂) and gelling (the reaction of a polyol with polyisocyanate).

Disadvantages in using conventional amine catalysts (for example, bisdimethylaminoethylether) in polyurethane foam production include: the occurrence of safety and toxicity problems due to their high volatility, resulting in airborne vapors thought to contribute to glaucopsia, also known as blue haze or halovision, which is a temporary disturbance for vision clarity; fogging of automotive windshields due to automotive interior foams produced from these catalysts; and malodorous properties.

In addition, many amine catalysts are also unstable with certain blowing agents, and in particular with the newer, low global-warming-potential (GWP) halogenated olefin blowing agents such as trans-1-chloro-3,3,3-trifluoropropene (known as 1233zd(E)) or cis-1,1,1,3,3,3-hexafluoro-2-butene (known as 1366mzz(Z)) due to their activated double bonds which can react with the amines. Various attempts have been made to improve the shelf life of blends containing amines and halogenated olefin blowing agents without affecting their ability to catalyze polyurethane foam formulation at a reasonable rate. Most of these attempts center around using amines that are deactivated in one way or another (e.g. sterically hindered or bonded with electron withdrawing groups) or by including additives to prevent their reaction with the halogenated olefin blowing agent (see, e.g., U.S. Pat. No. 10,023,681, US20150266994A1, US20160130416A1, U.S. Pat. Nos. 9,550,854, 9,556,303B2, 10,308,783B2, 9,868,837B2, US20190177465A1). However, such attempts have yet to achieve blends that have shelf-life stability and catalytic activity that is comparable to blends containing amines and standard non-halogenated blowing agents.

Thus, there is a continuing need for the development of new amine catalysts for use in producing rigid or flexible polyurethane foam and other polyurethane materials which may be combined with the newer, low global-warming-potential (GWP) halogenated olefin blowing agents above to form a blend having acceptable catalytic activity and an improved shelf life over the current conventional amine catalysts.

### SUMMARY

The present disclosure provides a polyurethane formulation comprising an acid-blocked pyrrolidine catalyst, a halogenated olefin compound, a compound containing an isocyanate functional group and an active hydrogen-containing compound.

According to another embodiment, there is provided a catalyst package for use in, for example but without limitation, forming a polyurethane material comprising an acid-blocked pyrrolidine catalyst and a halogenated olefin compound.

In yet another embodiment, there is provided a method of forming a polyurethane material comprising contacting a compound containing an isocyanate functional group, an active hydrogen-containing compound and optional auxiliary components in the presence of an acid-blocked pyrrolidine catalyst and a halogenated olefin compound.

### BRIEF DESCRIPTION OF THE DRAWINGS

FIG. 1 depicts the tack free times pre- and post-storage at 50° C. for polyurethane foams produced using acid-blocked industry standard catalysts as well as the inventive acid-blocked pyrrolidine catalysts. FIG. 2 depicts the stability of the polyurethane foam produced using the inventive acid-blocked pyrrolidine catalysts is good, with only a small drift in reactivity after aging the formulation for 6 weeks at 50° C. FIG. 3 illustrates that the drift (i.e., the change in cream time and string gel) of the polyurethane foam produced using the inventive acid-blocked pyrrolidine catalysts was not greater than 60% after 6 weeks of 50° C. storage, thereby demonstrating the unexpectedly superior stability of the presently claimed acid blocked catalysts as a polyurethane catalyst. FIG. 4 illustrates that the drift of the polyurethane foam produced using the comparative catalyst was as high as 260% after 6 weeks of storage at 50° C.

### DETAILED DESCRIPTION

The following terms shall have the following meanings:

The term “comprising” and derivatives thereof are not intended to exclude the presence of any additional component, step or procedure, whether or not the same is disclosed herein. In order to avoid any doubt, all compositions claimed herein through use of the term “comprising” may include any additional additive or compound, unless stated to the contrary. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e. to at least one) of the grammatical objects of the article. By way of example, “a catalyst” means one catalyst or more than one catalyst. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

The term “about” as used herein can allow for a degree of variability in a value or range, for example, it may be within 10%, within 5%, or within 1% of a stated value or of a stated limit of a range.

Values expressed in a range format should be interpreted in a flexible manner to include not only the numerical values explicitly recited as the limits of the range, but to also include all of the individual numerical values or sub-ranges encompassed within that range as if each numerical value and sub-range is explicitly recited. For example, a range such as from 1 to 6, should be considered to have specifically disclosed sub-ranges, such as, from 1 to 3, from 2 to 4, from 3 to 6, etc., as well as individual numbers within that range, for example, 1, 2, 3, 4, 5, and 6. This applies regardless of the breadth of the range.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

The term “substantially free” refers to a composition in which a particular compound or moiety is present in an amount that has no material effect on the composition. In some embodiments, “substantially free” may refer to a composition in which the particular compound or moiety is present in the composition in an amount of less than 2% by weight, or less than 1% by weight, or less than 0.5% by weight, or less than 0.1% by weight, or less than 0.05% by weight, or even less than 0.01% by weight based on the total weight of the composition, or that no amount of that particular compound or moiety is present in the respective composition.

Where substituent groups are specified by their conventional chemical formula, written from left to right, they equally encompass the chemically identical substituents that would result from writing the structure from right to left, for example, —CH₂O— is equivalent to —OCH₂—.

The term “alkyl” refers to straight chain or branched chain saturated hydrocarbon groups having from 1 to 10 carbon atoms. In some embodiments, alkyl substituents may be lower alkyl groups. The term “lower” refers to alkyl groups having from 1 to 6 carbon atoms. Examples of “lower alkyl groups” include, but are not limited to, methyl, ethyl, n-propyl, i-propyl, butyl, and pentyl groups.

The term “halogenated olefin” refers to an olefin compound or moiety which may include fluorine, chlorine, bromine or iodine.

The term “optional” or “optionally” means that the subsequently described event or circumstance may or may not occur, and that the description includes instances where said event or circumstance occurs and instances where it does not.

The present disclosure is generally directed to novel acid-blocked pyrrolidine catalysts and their use in polyurethane formulations which may include a compound containing an isocyanate functional group, an active hydrogen-containing compound and a halogenated olefin compound as a blowing agent. The present disclosure is also directed to rigid or flexible polyurethane foam or other polyurethane material made from a formulation comprising an acid-blocked pyrrolidine catalyst as described herein, a compound containing an isocyanate functional group, an active hydrogen-containing compound and a halogenated olefin compound as a blowing agent. The term “polyurethane” as used herein, is understood to encompass pure polyurethane, polyurethane polyurea, and pure polyurea. It has been surprisingly found combining a halogenated olefin compound blowing agent with an acid-blocked pyrrolidine catalyst according to the present disclosure, in place of a substantial portion of, or in place of all of, conventional amine catalysts, leads to a blend having improved shelf-life stability and catalytic activity.

According to one embodiment, the acid-blocked pyrrolidine catalyst is one or more catalysts represented by at least one of formula (1)

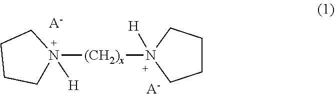

or formula (2)

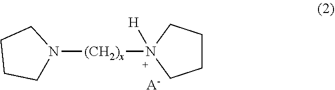

where x is an integer from 1 to 10 and A is an ion of an acidic compound, wherein the acidic compound has a formula (OH)ₙ—R—(COOH)ₘ where R is hydrogen, an alkyl, alkenyl, cycloaliphatic, aromatic, or alkylaromatic group, n and m are integers between 0 and 3 with the proviso that n+m≥1 and when n=1 and m=0, R is aromatic or alkylaromatic.

According to one embodiment, x is an integer from 1 to 9 or 1 to 8 or 1 to 7 or 1 to 6 or 1 to 5 or 1 to 4. In one particular embodiment, x is 2, 3 or 4. In another embodiment, x is an integer such that the (CH₂)ₓ group is a lower alkyl group.

According to another embodiment of the present disclosure, each A has from 1 to 10 carbon atoms and A is an ion of a carboxylic acid, a dicarboxylic acid, a tricarboxylic acid, a phenolic acid, a substituted phenolic acid or a hydroxy substituted derivative thereof.

Examples of R alkyl groups include, but are not limited to, methyl, ethyl, n-propyl, iso-propyl, propyl, butyl, iso-butyl, phenyl, ethylenyl, n-amyl, n-decyl or 2 ethylhexyl. While the aforementioned alkyl groups may comprise two available substitution sites, it is contemplated that additional hydrogens on the hydrocarbon could be replaced with further carboxyl and/or hydroxyl groups.

Particular compounds that may be used as component A include, but are not limited to, a hydroxyl-carboxylic acid, a di-carboxylic acid, formic acid, acetic acid, malonic acid, glutaric acid, maleic acid, glycolic acid, lactic acid, 2-hydroxybutyric acid, citric acid, AGS acid, phenol, cresol, hydroquinone, or combinations thereof. AGS acid is a mixture of dicarboxylic acids (i.e., adipic acid, glutaric acid, and succinic acid) that is obtained as a by-product of the oxidation of cyclohexanol and/or cyclohexanone in the adipic acid manufacturing process. Suitable AGS acid that may be used as component A include RHODIACID® acid (available from Solvay S.A.), DIBASIC acid (available from Invista S.a.r.l), FLEXATRAC™-AGS-200 acid (available from Ascend Performance Materials LLC), and glutaric acid, technical grade (AGS) (available from Lanxess A.G.).

In one embodiment, the acid-blocked pyrrolidine catalysts of formula (1) and (2) may be prepared in situ in the polyurethane formulation by adding the pyrrolidine and compound having a formula (OH)ₙ—R—(COOH)ₘ separately to the polyurethane formulation, while in other embodiments, the acid-blocked pyrrolidine catalysts above may be prepared prior to addition to the polyurethane formulation.

According to another embodiment, the acid-blocked pyrrolidine catalysts of formula (1) or (2) may be combined with a pyrrolidine catalyst having the formula (3) to form a catalyst mixture.

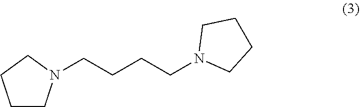 [SMILES: C1CCN(CCCCN2CCCC2)C1]

The pyrrolidine catalyst having the formula (3) may be combined with the acid-blocked pyrrolidine catalysts of formula (1) or (2) (or (1) and (2)) in amounts ranging from about 0.1% by weight to about 99.9% by weight, based on the total weight of the catalyst mixture. In another embodiment, the pyrrolidine catalyst having the formula (3) may be combined with the acid-blocked pyrrolidine catalysts of formula (1) or (2) (or (1) and (2)) in amounts ranging from about 1% by weight to about 90% by weight, or from about 10% by weight to about 80% by weight, or from about 20% by weight to about 70% by weight or from about 30% by weight to about 60% by weight or from about 40% by weight to about 50% by weight, based on the total weight of the catalyst mixture.

According to some embodiments, the acid-blocked pyrrolidine catalysts of formula (1) and/or (2) (and optionally the pyrrolidine catalyst of the formula (3)) may be used alone in forming the polyurethane foam or material. In still other embodiments, the catalysts above may be combined with an amine catalyst containing at least one tertiary amine group and/or a non-amine catalyst in forming the polyurethane foam or material. In embodiments in which the acid-blocked pyrrolidine catalysts (1) and/or (2) are combined with an amine catalyst containing at least one tertiary amine group and/or a non-amine catalyst, the weight ratio of the acid-blocked pyrrolidine catalysts of formula (1) and/or (2) to the amine catalyst containing at least one amine group and/or the non-amine catalyst is at least 1:1, and in some embodiments, at least 1.5:1 and in still other embodiments at least 2:1 and in further embodiments at least 5:1, while in still further embodiments at least 10:1. In still other embodiments, the weight ratio of the acid-blocked pyrrolidine catalyst of formula (1) and/or (2) to the amine catalyst containing at least one amine group and/or the non-amine catalyst is from 0.1:99.9 to 99.9:0.1, and in still other embodiments from 1:99 to 99:1, and in still other embodiments from 5:95 to 95:5, and in further embodiments from 10:90 to 90:10, while in still further embodiments from 25:75 to 75:25.

Representative amine catalysts containing at least one tertiary group include, but are not limited to, bis-(2-dimethylaminoethyl)ether (JEFFCAT® ZF-20 catalyst), N,N,N′-trimethyl-N′-hydroxyethylbisaminoethyl ether (JEFFCAT® ZF-10 catalyst), N-(3-dimethylaminopropyl)-N,N-diisopropanolamine (JEFFCAT® DPA catalyst), N,N-dimethylethanolamine (JEFFCAT® DMEA catalyst), triethylene diamine (JEFFCAT® TEDA catalyst), blends of N,N-dimethylethanolamine ethylene diamine (such as JEFFCAT® TD-20 catalyst), N,N-dimethylcyclohexylamine (JEFFCAT® DMCHA catalyst), benzyldimethylamine (JEFFCAT® BDMA catalyst), pentamethyldiethylenetriamine (JEFFCAT® PMDETA catalyst), N,N,N′,N″,N″-pentamethyldipropylenetriamine (JEFFCAT® ZR-40 catalyst), N,N-bis(3-dimethylaminopropyl)-N-isopropanolamine (JEFFCAT® ZR-50 catalyst), N′-(3-(dimethylamino)propyl-N,N-dimethyl-1,3-propanediamine (JEFFCAT® Z130 catalyst), 2-(2-dimethylaminoethoxy)ethanol (JEFFCAT® ZR-70 catalyst), N,N,N-trimethylaminoethyl-ethanolamine (JEFFCAT® Z-110 catalyst), N-ethylmorpholine (JEFFCAT® NEM catalyst), N-methylmorpholine (JEFFCAT® NMM catalyst), 4-methoxyethylmorpholine, N,N′dimethylpiperzine (JEFFCAT® DMP catalyst), 2,2′-dimorpholinodiethylether (JEFFCAT® DMDEE catalyst), 1,3,5-tris(3-(dimethylamino)propyl)-hexahydro-s-triazine (JEFFCAT® TR-90 catalyst), 1-propanamine, 3-(2-(dimethylamino)ethoxy), substituted imidazoles such as 1,2-dimethlyimidazol and 1-methyl-2-hydroxyethylimidazole, N,N′-dimethylpiperazines or bis-substituted piperazines such aminoethylpiperazine, N,N′,N′-trimethyl aminoethylpiperazine or bis-(N-methyl piperazine)urea, N-methylpyrrolidines and substituted methylpyrrolidines such as 2-aminoethyl-N-methylpyrrolidine or bis-(N-methylpyrrolidine)ethyl urea, 3-dimethylaminopropylamine, N,N,N″,N″-tetram ethyldipropylenetriamine, tetramethylguanidine, 1,2-bis-diisopropanol. Other examples of amine catalysts include N-alkylmorpholines, such as N-methylmorpholine, N-ethylmorpholine, N-butylmorpholine and dimorpholinodiethylether, N,N′-dimethylaminoethanol, N,N-dimethylamino ethoxyethanol, bis-(dimethylaminopropyl)-amino-2-propanol, bis-(dimethylamino)-2-propanol, bis-(N,N-dimethylamino)ethylether; N,N,N′-trimethyl-N′hydroxyethyl-bis-(aminoethyl)ether, N,N-dimethyl amino ethyl -N′-methyl amino ethanol and tetramethyliminobispropylamine. The aforementioned JEFFCAT® catalysts are available from Huntsman Petrochemical LLC, The Woodlands, Texas.

Other amine catalysts which may be used in the present disclosure may be found in Appendix D in “Dow Polyurethanes Flexible Foams” by Herrington et al. at pages D.1-D.23 (1997), which is incorporated herein by reference. Further examples may be found in “JEFFCAT® Amine Catalysts for the Polyurethane Industry” version JCT-0910 which is incorporated herein by reference.

The non-amine catalyst is a compound (or mixture thereof) having catalytic activity for the reaction of an isocyanate group with a polyol or water, but is not a compound falling within the description of the amine catalyst above. Examples of such additional non-amine catalysts include, for example:

tertiary phosphines, such as trialkylphosphines and dialkylbenzylphosphines;

chelates of various metals, such as those which can be obtained from acetylacetone, benzoylacetone, trifluoroacetyl acetone, ethyl acetoacetate and the like, with metals such as Be, Mg, Zn, Cd, Pd, Ti, Zr, Sn, As, Bi, Cr, Mo, Mn, Fe, Co and Ni;

metal carboxylates such as potassium acetate and sodium acetate;

acidic metal salts of strong acids, such as ferric chloride, stannic chloride, stannous chloride, antimony trichloride, bismuth nitrate and bismuth chloride;

strong bases, such as alkali and alkaline earth metal hydroxides, alkoxides and phenoxides;

alcoholates and phenolates of various metals, such as Ti(OR⁶)₄, Sn(OR⁶)₄ and Al(OR⁶)₃ where R⁶ is alkyl or aryl, and the reaction products of the alcoholates with carboxylic acids, beta-diketones and 2-(N,N-dialkylamino) alcohols;

alkaline earth metal, Bi, Pb, Sn or Al carboxylate salts; and tetravalent tin compounds, and tri- or pentavalent bismuth, antimony or arsenic compounds.

The acid-blocked pyrrolidine catalysts of formula (1) and/or (2) may be used in a catalytically effective amount to catalyze the reaction between a compound containing an isocyanate functional group and an active hydrogen-containing compound for making rigid or flexible polyurethane foam or other polyurethane materials. A catalytically effective amount of the acid blocked pyrrolidine catalysts of formula (1) and/or (2) may range from about 0.01-15 parts per 100 parts of active hydrogen-containing compound, and in some embodiments from about 0.05-12.5 parts per 100 parts of active hydrogen-containing compound, and in even further embodiments from about 0.1-7.5 parts per 100 parts of active hydrogen-containing compound, and yet in even further embodiments from about 0.5-5 parts per 100 parts of active hydrogen-containing compound.

In one embodiment, the compound containing an isocyanate functional group is a polyisocyanate and/or an isocyanate-terminated prepolymer.

Polyisocyanates include those represented by the general formula Q(NCO)a where a is a number from 2-5, such as 2-3 and Q is an aliphatic hydrocarbon group containing 2-18 carbon atoms, a cycloaliphatic hydrocarbon group containing 5-10 carbon atoms, an araliphatic hydrocarbon group containing 8-13 carbon atoms, or an aromatic hydrocarbon group containing 6-15 carbon atoms.

Examples of polyisocyanates include, but are not limited to, ethylene diisocyanate; 1,4-tetramethylene diisocyanate; 1,6-hexamethylene diisocyanate; 1,12-dodecane diisocyanate; cyclobutane-1,3-diisocyanate; cyclohexane-1,3- and 1,4-diisocyanate, and mixtures of these isomers; isophorone diisocyanate; 2,4- and 2,6-hexahydrotoluene diisocyanate and mixtures of these isomers; dicyclohexylmethane-4,4′-diisocyanate (hydrogenated MDI, or HMDI); 1,3- and 1,4-phenylene diisocyanate; 2,4- and 2,6-toluene diisocyanate and mixtures of these isomers (TDI); diphenylmethane-2,4′-and/or -4,4′-diisocyanate (MDI); naphthylene-1,5-diisocyanate; triphenylmethane-4,4′,4″-triisocyanate; polyphenyl-polymethylene-polyisocyanates of the type which may be obtained by condensing aniline with formaldehyde, followed by phosgenation (crude MDI); norbornane diisocyanates; m- and p-isocyanatophenyl sulfonylisocyanates; perchlorinated aryl polyisocyanates; modified polyisocyanates containing carbodiimide groups, urethane groups, allophnate groups, isocyanurate groups, urea groups, or biruret groups; polyisocyanates obtained by telomerization reactions; polyisocyanates containing ester groups; and polyisocyanates containing polymeric fatty acid groups. Those skilled in the art will recognize that it is also possible to use mixtures of the polyisocyanates described above.

Isocyanate-terminated prepolymers may also be employed in the preparation of the polyurethane. Isocyanate-terminated prepolymers may be prepared by reacting an excess of polyisocyanate or mixture thereof with a minor amount of an active-hydrogen containing compound as determined by the well-known Zerewitinoff test.

In another embodiment, the active hydrogen-containing compound is a polyol. Polyols suitable for use in the present disclosure include, but are not limited to, polyalkylene ether polyols, polyester polyols, polymer polyols, a non-flammable polyol such as a phosphorus-containing polyol or a halogen-containing polyol. Such polyols may be used alone or in suitable combination as a mixture.

Polyalkylene ether polyols include poly(alkylene oxide) polymers such as poly(ethylene oxide) and polypropylene oxide) polymers and copolymers with terminal hydroxyl groups derived from polyhydric compounds, including diols and triols; for example, ethylene glycol, propylene glycol, 1,3-butane diol, 1,4-butane diol, 1,6-hexane diol, neopentyl glycol, diethylene glycol, dipropylene glycol, pentaerythritol, glycerol, diglycerol, trimethylol propane, and similar low molecular weight polyols.

Polyester polyols include, but are not limited to, those produced by reacting a dicarboxylic acid with an excess of a diol, for example, adipic acid with ethylene glycol or butanediol, or reaction of a lactone with an excess of a diol such as caprolactone with propylene glycol.

In addition to polyalkylene ether polyols and polyester polyols, polymer polyols are also suitable for use in the present disclosure. Polymer polyols are used in polyurethane materials to increase resistance to deformation, for example, to improve the load-bearing properties of the foam or material. Examples of polymer polyols include, but are not limited to, graft polyols or polyurea modified polyols (Polyharnstoff Dispersion polyols). Graft polyols comprise a triol in which vinyl monomers are graft copolymerized. Suitable vinyl monomers include, for example, styrene, or acrylonitrile. A polyurea modified polyol is a polyol containing a polyurea dispersion formed by the reaction of a diamine and a diisocyanate in the presence of a polyol. A variant of polyurea modified polyols are polyisocyanate poly addition (PIPA) polyols, which are formed by the in situ reaction of an isocyanate and an alkanolamine in a polyol.

The non-flammable polyol may, for example, be a phosphorus-containing polyol obtainable by adding an alkylene oxide to a phosphoric acid compound. A halogen-containing polyol may, for example, be those obtainable by ring-opening polymerization of epichlorohydrin or trichlorobutylene oxide.

The polyurethane formulation may also contain one or more halogenated olefin compounds that serve as a blowing agent. The halogenated olefin compound comprises at least one haloalkene (e.g, fluoroalkene or chlorofluoroalkene) comprising from 3 to 4 carbon atoms and at least one carbon-carbon double bond. Suitable compounds may include hydrohaloolefins such as trifluoropropenes, tetrafluoropropenes (e.g., tetrafluoropropene (1234)), pentafluoropropenes (e.g., pentafluoropropene (1225)), chlorotrifloropropenes (e.g., chlorotrifloropropene (1233)), chlorodifluoropropenes, chlorotrifluoropropenes, chlorotetrafluoropropenes, hexafluorobutenes (e.g., hexafluorobutene (1336)), or combinations thereof. In certain embodiments, the tetrafluoropropene, pentafluoropropene, and/or chlorotrifloropropene compounds have no more than one fluorine or chlorine substituent connected to the terminal carbon atom of the unsaturated carbon chain (e.g., 1,3,3,3-tetrafluoropropene (1234ze); 1,1,3,3-tetrafluoropropene, 1,2,3,3,3 -pentafluoropropene (1225ye), 1,1,1-trifluoropropene, 1,2,3,3,3-pentafluoropropene, 1,1,1,3,3-pentafluoropropene (1225zc), 1,1,2,3,3-pentafluoropropene (1225yc), (Z)-1,1, 1,2,3-pentafluoropropene (1225yez), 1-chloro-3,3,3-trifluoropropene (1233zd), 1,1,1,4,4,4-hexafluorobut-2-ene (1336mzzm), or combinations thereof).

Other blowing agents that may be used in combination with the halogenated olefin compounds described above include air, nitrogen, carbon dioxide, hydrofluorocarbons (“HFCs”), alkanes, alkenes, mono-carboxylic acid salts, ketones, ethers, or combinations thereof. Suitable HFCs include 1,1-difluoroethane (HFC-152a), 1,1,1,2-tetrafluoroethane (HFC-134a), pentafluoroethane (HFC-125), 1,1,1,3,3 -pentafluoropropane (HFC-245fa), 1,1,1,3,3-pentaflurobutane (HFC-365mfc) or combinations thereof. Suitable alkanes and alkenes include n-butane, n-pentane, isopentane, cyclopentane, 1-pentene, or combinations thereof. Suitable mono-carboxylic acid salts include methyl formate, ethyl formate, methyl acetate, or combinations thereof. Suitable ketones and ethers include acetone, dimethyl ether, or combinations thereof.

In addition, the polyurethane formulation may optionally include one or more auxiliary components. Examples of auxiliary components include, but are not limited to, cell stabilizers, surfactants, chain extenders, pigments, fillers, flame retardants, thermally expandable microspheres, water, thickening agents, smoke suppressants, reinforcements, antioxidants, UV stabilizers, antistatic agents, infrared radiation absorbers, dyes, mold release agents, antifungal agents, biocides or any combination thereof.

Cell stabilizers may include, for example, silicon surfactants or anionic surfactants. Examples of suitable silicon surfactants include, but are not limited to, polyalkylsiloxane, polyoxyalkylene polyol-modified dimethylpolysiloxane, alkylene glycol-modified dimethylpolysiloxane, or any combination thereof.

Suitable surfactants (or surface-active agents) include emulsifiers and foam stabilizers, such as silicone surfactants known in the art, for example, polysiloxanes, as well as various amine salts of fatty acids, such as diethylamine oleate or diethanolamine stearate, as well as sodium salts of ricinoleic acids.

Examples of chain extenders include, but are not limited to, compounds having hydroxyl or amino functional groups, such as glycols, amines, diols, and water. Further non-limiting examples of chain extenders include ethylene glycol, propylene glycol, 1,4-butanediol, 1,3-butanediol, 1,5-pentanediol, 1,6-hexanediol, 1,10-decanediol, 1,12-dodecanediol, ethoxylated hydroquinone, 1,4-cyclohexanediol, N-methylethanolamine, N-methylisopropanolamine, 4-aminocyclo-hexanol, 1,2-diaminoethane, or any mixture thereof.

Pigments may be used to color code the polyurethane materials during manufacture, to identify product grade, or to conceal yellowing. Pigments may include any suitable organic or inorganic pigments. For example, organic pigments or colorants include, but are not limited to, azo/diazo dyes, phthalocyanines, dioxazines, or carbon black. Examples of inorganic pigments include, but are not limited to, titanium dioxide, iron oxides or chromium oxide.

Fillers may be used to increase the density and load bearing properties of polyurethane foam or material. Suitable fillers include, but are not limited to, barium sulfate, carbon black or calcium carbonate.

Flame retardants can be used to reduce flammability. For example, such flame retardants include, but are not limited to, chlorinated phosphate esters, chlorinated paraffins or melamine powders.

Thermally expandable microspheres include those containing a (cyclo)aliphatic hydrocarbon. Such microspheres are generally dry, unexpanded or partially unexpanded microspheres consisting of small spherical particles with an average diameter of typically 10 to 15 micron. The sphere is formed of a gas proof polymeric shell (e.g. consisting of acrylonitrile or PVDC), encapsulating a minute drop of a (cyclo)aliphatic hydrocarbon, e.g. liquid isobutane. When these microspheres are subjected to heat at an elevated temperature level (e.g. 150° C. to 200° C.) sufficient to soften the thermoplastic shell and to volatilize the (cyclo)aliphatic hydrocarbon encapsulated therein, the resultant gas expands the shell and increases the volume of the microspheres. When expanded, the microspheres have a diameter 3.5 to 4 times their original diameter as a consequence of which their expanded volume is about 50 to 60 times greater than their initial volume in the unexpanded state. Examples of such microspheres are the EXPANCEL®-DU microspheres which are marketed by AKZO Nobel Industries of Sweden.

The methods for producing a polyurethane material from a polyurethane formulation according to the present disclosure are well known to those skilled in the art and can be found in, for example, U.S. Pat. Nos. 5,420,170, 5,648,447, 6,107,359, 6,552,100, 6,737,471 and 6,790,872, the contents of which are hereby incorporated by reference. Various types of polyurethane materials can be made, such as rigid foams, flexible foams, semi-flexible foams, microcellular elastomers, backings for textiles, spray elastomers, cast elastomers, polyurethane-isocyanurate foams, reaction injection molded polymers, structural reaction injection molded polymers and the like.

A non-limiting example of a general flexible polyurethane foam formulation having a 15-150 kg/m³ density (e.g. automotive seating) containing the acid-blocked pyrrolidine catalyst of formula (1) and (2) may comprise the following components in parts by weight (pbw):

| Flexible Foam Formulation | pbw |
|---|---|
| Polyol | 20-100 |
| Surfactant | 0.3-3 |
| Blowing Agent | 1-6 |
| Crosslinker | 0-3 |
| Acid-blocked pyrrolidine catalyst | 0.2-2.5 |
| Isocyanate Index | 70-115 |

A non-limiting example of a general rigid polyurethane foam formulation having a 15-70 kg/m³ density containing the acid-blocked pyrrolidine catalyst of formula (1) or (2) may comprise the following components in parts by weight (pbw):

| Rigid Foam Formulation | Pbw |
|---|---|
| Polyol | 100 |
| Surfactant | 1-3 |
| Blowing Agent | 20-40 |
| Water | 0-3 |
| Acid-blocked pyrrolidine catalyst | 0.5-3 |
| Isocyanate Index | 80-400 |

The amount of the compound containing an isocyanate functional group is not limited, but will generally be within those ranges known to one skilled in the art. An exemplary range given above is indicated by reference to Isocyanate Index which is defined as the number of equivalents of isocyanate divided by the total number of equivalents of active hydrogen, multiplied by 100.

Thus, in yet another embodiment, the present disclosure provides a method for producing a polyurethane material which comprises contacting the compound containing an isocyanate functional group, an active hydrogen-containing compound, halogenated olefin and optional auxiliary components in the presence of the acid-blocked pyrrolidine catalysts according to the present disclosure.

In one particular embodiment, the polyurethane material is a rigid or flexible foam prepared by bringing together at least one polyol and at least one polyisocyanate in the presence of the acid-blocked pyrrolidine catalyst of formula (1) and/or (2) and halogenated olefin compound to form a reaction mixture and subjecting the reaction mixture to conditions sufficient to cause the polyol to react with the polyisocyanate. The polyol, polyisocyanate, acid-blocked pyrrolidine catalyst and halogenated olefin compound may be heated prior to mixing them and forming the reaction mixture. In other embodiments, the polyol, polyisocyanate, acid-blocked pyrrolidine catalyst and halogenated olefin compound are mixed at ambient temperature (for e.g. from about 15°-40° C.) and heat may be applied to the reaction mixture, but in some embodiments, applying heat may not be necessary. The polyurethane foam may be made in a free rise (slabstock) process in which the foam is free to rise under minimal or no vertical constraints. Alternatively, molded foam may be made by introducing the reaction mixture in a closed mold and allowing it to foam within the mold. The particular polyol and polyisocyanate are selected with the desired characteristics of the resulting foam. Other auxiliary components useful in making polyurethane foams, such as those described above, may also be included to produce a particular type of foam.

According to another embodiment, a polyurethane material may be produced in a one-step process in which an A-side reactant is reacted with a B-side reactant. The A-side reactant may comprise a polyisocyanate while the B-side reactant may comprise a polyol, the acid-blocked pyrrolidine catalyst and halogenated olefin compound. In some embodiments, the A-side and/or B-side may also optionally contain other auxiliary components such as those described above.

The polyurethane materials produced may be used in a variety of applications, such as, a precoat; a backing material for carpet; building composites; insulation; spray foam insulation; applications requiring use of impingement mix spray guns; urethane/urea hybrid elastomers; vehicle interior and exterior parts such as bed liners, dashboards, door panels, and steering wheels; flexible foams (such as furniture foams and vehicle component foams); integral skin foams; rigid spray foams; rigid pour-in-place foams; coatings; adhesives; sealants; filament winding; and other polyurethane composite, foams, elastomers, resins, and reaction injection molding (RIM) applications

The present disclosure will now be further described with reference to the following non-limiting examples.

### EXAMPLES

### Example 1

Polyurethane foams were made from MDI and polyol resin blends (as set forth in Table 1), wherein the Catalyst in the polyol resin blends was selected from various state of the art amine catalysts (JEFFCAT® ZF-10, ZF-20, Z-110, Z-130, ZR-70, as described earlier, which have been mixed with glutaric acid or formic acid) or an example of the inventive acid-blocked pyrrolidine catalyst as set forth herein (“XP CAT”), which is represented by a mixture of catalysts of formulas 1 and 2 with both formulas having x=4. In one sample of XP CAT, A (as represented in formulas 1 and 2) is an ion of formic acid. In a second sample of XP CAT, A (as represented in formulas 1 and 2) is an ion of glutaric acid.

TABLE 1

| Component | Percent |
|---|---|
| TEROL ® 649 | 40.84 |
| JEFFOL ® R-425-X | 14.78 |
| JEFFOL ® SG-522 | 7.88 |
| Flame retardant A | 6.80 |
| Flame retardant B | 11.00 |
| Silicone surfactant | 1.00 |
| Water | 1.70 |
| Catalyst | 5.00 |
| Blowing agent | 11.00 |
| Total | 100.00 |

As noted, Table 1 shows the components of the polyol resin blends. TEROL® 649 polyol is a modified aromatic polyester polyol. JEFFOL® R-425-X polyether polyol is an amine-based polyether polyol. JEFFOL® SG-522 polyol is a sucrose-based polyol. JEFFOL® polyether polyol products and TEROL® aromatic polyester polyol products are commercially available from Huntsman Corporation (The Woodlands, Texas). Flame retardant A was a tetrabromophthalate diol, commercially available as PHT4-Diol™ reactive halogenated flame retardant from LANXESS AG (Cologne, Germany). Flame retardant B was a chlorinated phosphate ester. The silicone surfactant used was Dabco® DC-193 silicone surfactant which is commercially available from Evonik Industries AG (Essen, Germany). The blowing agent used was a halogenated olefinic blowing agent manufactured by Honeywell Corporation under the name SOLSTICE® LBA blowing agent.

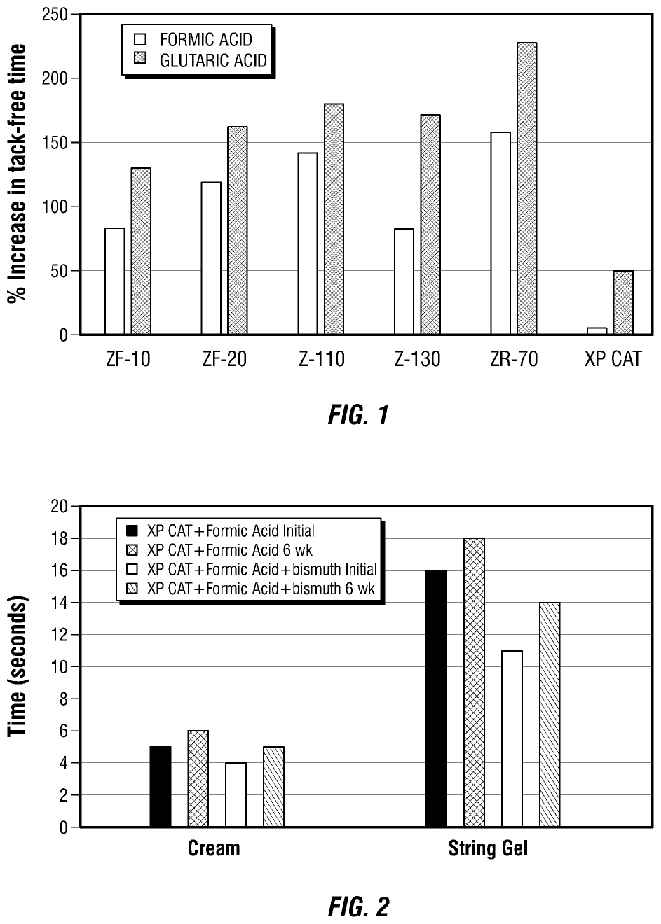

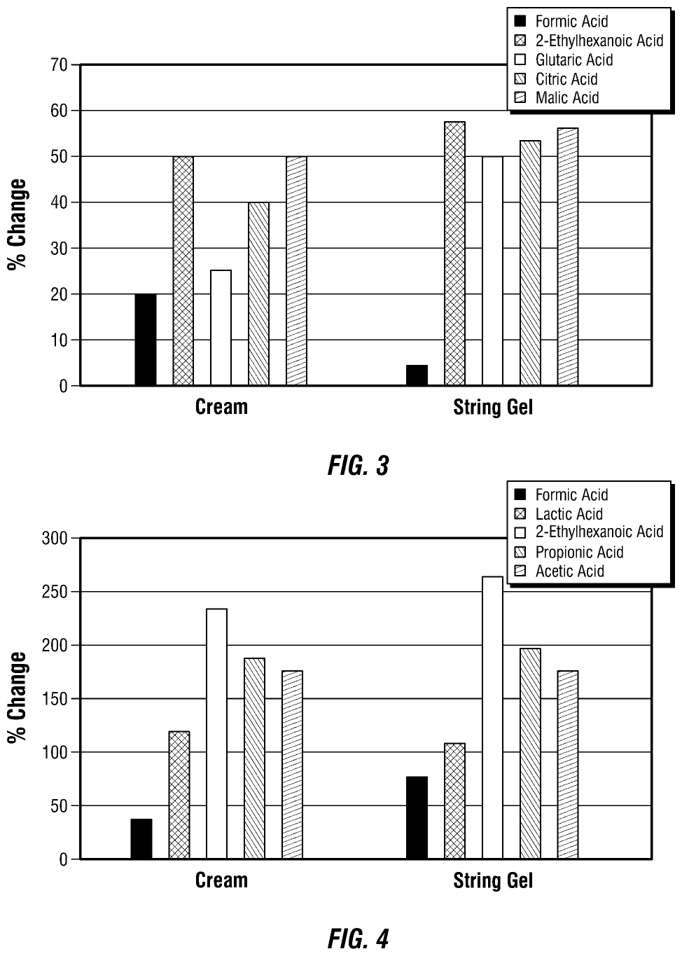

Following a procedure such as that of ASTM D7487-18, the foams were mixed vigorously for 4 seconds in a cup using 50 g of polyol resin blend and 50 g MDI and then the foam profile was measured using a stopwatch. The “tack-free time” of the foams, as they were formed, was measured initially and after 6 weeks of storage as an indicator of the stability of the system, the results for which are presented in FIG. 1. A polyol blend that is unstable will inherently produce foams with slower tack-free times as the blowing agent and/or catalysts are deactivated by reacting together. As is evident from FIG. 1, the blends containing the inventive acid-blocked pyrrolidine catalyst (“XP CAT”) were much more stable than the mixtures of the state of the art catalysts and formic or glutaric acid. This was unexpected, since the pyrrolidinyl nitrogen of the XP CAT has a similar or higher pKa to that of the aminomethyl moieties of standard catalysts (Table 2) and amines with higher pKa values are expected to be more reactive with halogenated olefinic blowing agents. In fact, given the high nucleophilicity of the pyrrolidinyl group that has been experimentally measured by Mayr et. al. (J. Org. Chem. 2007, 72, 3679-3688), it is completely unexpected that these inventive compounds would be more stable with the halogenated olefinic blowing agents than their linear alkylamino analogues.

TABLE 2

| Amine | pKa | ref |
|---|---|---|
| JEFFCAT ® ZF-20 | 9.12 ± 0.28 | 1 |
| PMDETA | 9.1 | 3 |
| 1, XP CAT | 10.8 ± 0.20 | 1 |
| dimethylcyclohexylamine | 10.1 | 4 |
| JEFFCAT ® Z-130 | 10.4 ± 0.19 | 1 |
| JEFFCAT ® ZR-70 | 9.1 | 3 |
| N-methylpyrrolidine | 10.46 | 2 |
| JEFFCAT ® Z-110 | 9.18 | 5 |

¹Calculated using Advanced Chemistry Development (ACD/Labs) Software VI1.02 (© 1994-2019 ACD/Labs)
²CRC Handbook of Chemistry and Physics
³U.S. Pat. No. 9,051,442
⁴J. Org. Chem. 1961, 26, 3, 779-782
⁵J. Chem. Eng. Data 2016, 61, 247-254

### Example 2

Many acid-blocked amine catalysts are not compatible in the presence of halogenated olefinic blowing agents when stored with metal co-catalysts that are commonly used in polyurethane spray foam, typically forming solid precipitates in the polyol resin blend and inhibiting foam reactivity. The formulation from Table 1 was used to evaluate polyurethane foams, wherein the Catalyst in Table 1 comprised XP CAT and formic acid with and without a bismuth co-catalyst. Following a procedure such as that of ASTM D7487-18, the cream time and string gel times were measured for polyurethane foams produced immediately after blending such formulations (with and without bismuth) and again after s such formulations were aged for 6 weeks at 50° C. (both with and without bismuth) . In the systems with bismuth, BiCat® 8842 from Shepherd chemical was used at 0.5 wt % based on the total weight of the formulation in Table 1.

The cream time and string gel time measurements were taken following a procedure such as that of ASTM D7487-18.

FIG. 2 shows that with and without bismuth, the stability of the polyurethane foam is good, with only a small drift in reactivity after aging the formulation for 6 weeks at 50° C.

### Example 3

Using the same procedure as in Example 1, the storage stability of the XP CAT in combination with various acids (i.e., formic acid, 2-ethylhexanoic acid, glutaric acid, citric acid, and malic acid) as the “A” in formulas 1 and 2 was evaluated by measuring the change in cream time and string gel for polyurethane formulations prepared using polyol blends of the XP CAT and acids (as set forth in Table 1) shortly after preparing the polyol blends and also after aging the polyol blends for 6 weeks at 50 ° C. As seen in FIG. 3, the drift (i.e., the change in cream time and string gel) was not greater than 60% after 6 weeks of 50° C. storage. This demonstrates the unexpectedly superior stability of the presently claimed acid blocked catalysts as a polyurethane catalyst for systems using halogenated olefinic blowing agents.

### Example 4

Using the same procedure as in Example 1, the storage stability of a comparative catalyst, JEFFCAT® LE-30, in combination with various acids (i.e., formic acid, lactic acid, 2-ethylhexanoic acid, propionic acid, and acetic acid) was evaluated by measuring the change in cream time and string gel for polyurethane formulations prepared using polyol blends of the XP CAT and acids as the Catalyst (as set forth in Table 1) shortly after preparing the polyol blends and also after aging the polyol blends for 6 weeks at 50° C. As seen in FIG. 4, the drift was as high as 260% after 6 weeks of storage at 50° C. This further demonstrates the unexpectedly superior stability of the presently claimed acid blocked catalysts as a polyurethane catalyst for systems using halogenated olefinic blowing agents as compared with current state of the art catalysts.

What is claimed is:

1. A polyurethane formulation comprising: (i) an acid-blocked pyrrolidine catalyst represented by at least one of formula (1) and/or formula (2): 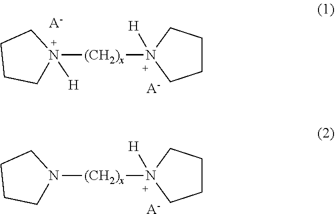
where x is an integer from 1 to 10 and A is an ion of an acidic compound, wherein the acidic compound has a formula (OH)ₙ—R—(COOH)ₘ where R is hydrogen, an alkyl, alkenyl, cycloaliphatic, aromatic, or alkylaromatic group, n and m are integers between 0 and 3, with the proviso that n+m≥1 and when n=1 and m=0, R is aromatic or alkylaromatic; (ii) a compound containing an isocyanate functional group; (iii) an active hydrogen-containing compound; and (iv) a halogenated olefin compound.

2. The polyurethane formulation of claim 1, wherein x is an integer from 1 to 4.

3. The polyurethane formulation of claim 1, wherein R is methyl, ethyl, n-propyl, iso-propyl, propyl, butyl, iso-butyl, n-amyl, n-decyl or 2 ethylhexyl.

4. The polyurethane formulation of claim 1, wherein the polyurethane formulation further comprises an amine catalyst containing at least one tertiary amine group and/or a non-amine catalyst.

5. A polyurethane formulation comprising: (i) an acid-blocked pyrrolidine catalyst represented by at least one of formula (1) and/or formula (2) 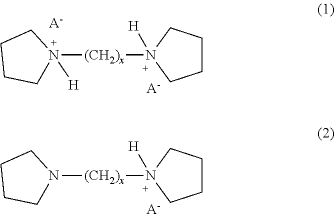
where x is an integer from 1 to 10 and A is an ion of an acidic compound, wherein the acidic compound has a formula (OH)ₙ—R—(COOH)ₘ where R is hydrogen, an alkyl, alkenyl, cycloaliphatic, aromatic, or alkylaromatic group, n and m are integers between 0 and 3, with the proviso that n+m≥1 and when n=1 and m=0, R is aromatic or alkylaromatic; (ii) a compound containing an isocyanate functional group; (iii) an active hydrogen-containing compound; (iv) a halogenated olefin compound; and (v) a pyrrolidine catalyst having the formula (3)

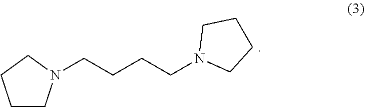 [SMILES: C1CCN(CCCCN2CCCC2)C1]

6. A catalyst package comprising: (i) an acid-blocked pyrrolidine catalyst represented by at least one of formula (1) and/or formula (2) 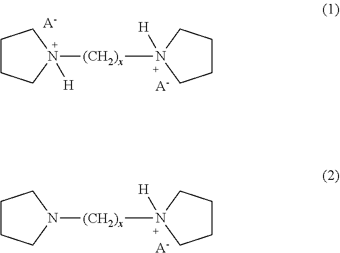
where x is an integer from 1 to 10 and A is an ion of an acidic compound, wherein the acidic compound has a formula (OH)ₙ—R—(COOH)ₘ where R is hydrogen, an alkyl, alkenyl, cycloaliphatic, aromatic, or alkylaromatic group, n and m are integers between 0 and 3, with the proviso that n+m≥1 and when n=1 and m=0, R is aromatic or alkylaromatic; and a halogenated olefin compound.

7. The catalyst package of claim 6, further comprising a pyrrolidine catalyst having the formula (3)

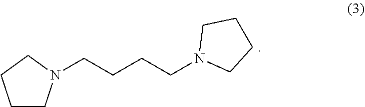 [SMILES: C1CCN(CCCCN2CCCC2)C1]

8. A method for producing a polyurethane material comprising contacting a compound containing an isocyanate functional group, an active hydrogen-containing compound and optional auxiliary components in the presence of an acid-blocked pyrrolidine catalyst represented by at least one of formula (1) and/or formula (2) 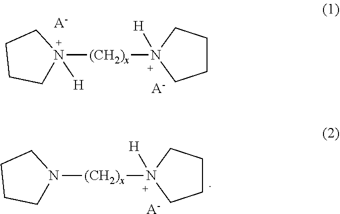
where x is an integer from 1 to 10 and A is an ion of an acidic compound, wherein the acidic compound has a formula (OH)ₙ—R—(COOH)ₘ where R is hydrogen, an alkyl, alkenyl, cycloaliphatic, aromatic, or alkylaromatic group, n and m are integers between 0 and 3, with the proviso that n+m≥1 and when n=1 and m=0, R is aromatic or alkylaromatic; and a halogenated olefin compound.

9. A polyurethane material produced according to the method of claim 8.

10. The polyurethane material of claim 9, wherein the polyurethane material is a rigid foam or a flexible foam.

11. The polyurethane material produced according to the method of claim 8 for use as a precoat, a backing material for carpet, a building composite, insulation, a spray foam insulation, a urethane/urea hybrid elastomers; in vehicle interior and exterior parts, a flexible foam, an integral skin foam, a rigid spray foam, a rigid pour-in-place foam; a coating; an adhesive, a sealant, or a filament winding.
